(19) United States
(12) Patent Application Publication
(10) Pub. No.: US 2025/0340523 A1
(43) Pub. Date: Nov. 6, 2025

(54) COMPOUNDS AND METHODS FOR INCREASING EFFICIENCY OF CARDIAC METABOLISM

(71) Applicant: IMBRIA PHARMACEUTICALS, INC., Boston, MA (US)

(72) Inventor: Edward P. Monaghan, Rancho Santa Margarita, CA (US)

(21) Appl. No.: 18/868,353

(22) PCT Filed: May 30, 2023
(86) PCT No.: PCT/US2023/023833

§ 371 Date: Nov. 22, 2024

Related U.S. Application Data
(60) Provisional application No. 63/348,775, filed on Jun. 3, 2022.

(51) Int. Cl. C07D 295/15

(52) U.S. Cl. CPC C07D 295/15

(57) ABSTRACT

The invention provides compositions for treating cardiovascular conditions and methods of increasing the efficiency of cardiac metabolism comprising the compound of formula (X).

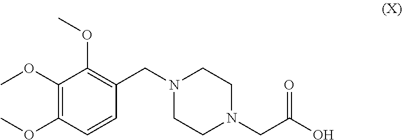 [SMILES: COc1ccc(CN2CCN(CC(=O)O)CC2)c(OC)c1OC]

### FIELD OF THE INVENTION

The invention relates to compositions and methods for treating cardiovascular conditions and increasing the efficiency of cardiac metabolism.

### BACKGROUND

Cardiovascular disease is the leading cause of death worldwide, accounting for ˜17.9 million deaths across the globe (WHO), and representing ˜32% of all global deaths. In coronary artery disease (CAD), the most common cardiovascular disease, blood flow to the heart muscle is reduced, usually due to accumulation of atherosclerotic plaque in the epicardial coronary arteries. CAD can lead to heart failure, a clinical syndrome due to a structural and/or functional abnormality of the heart that results in elevated intra-cardiac pressures and/or inadequate cardiac output at rest and/or during exercise. Most heart failure arises as a result of systolic and/or diastolic myocardial dysfunction, but heart failure can also be primarily a result of abnormalities of the endocardium, pericardium, valves, heart rhythm and/or cardiac conduction.

The heart is dependent on a continuous energy supply in the form of adenosine triphosphate (ATP) for normal systolic and diastolic performance. The amount of energy required to support contractile function including excitation-contraction coupling and diastolic active relaxation, basal metabolic processes, and maintenance of ionic homeostasis is derived almost exclusively by oxidative phosphorylation of substrates in the mitochondria. Mitochondria are sub-cellular organelles in which metabolites derived from carbon substrates such as glucose and fatty acids are oxidized to produce high-energy molecules. Glucose oxidation is a more efficient source of energy generation than fatty acids and other substrates (including ketone bodies). Specifically, glucose oxidation produces more ATP for the same amount of oxygen available. Accordingly, there is a marked energetic and mechanical efficiency advantage to shifting cardiac metabolism towards glucose oxidation.

Given this vast demand for ATP to maintain cardiac function coupled with relatively low ATP content of cardiomyocytes, it is unsurprising that there are functional consequences if there is a mismatch between ATP generation and demand, leading to energy deprivation, such as occurs in cardiac disease states. Moreover, as with any mechanical pump, only part of the energy invested is converted into external power. In certain types of heart disease, such as heart failure, ischemic heart disease, and diabetic cardiomyopathies, there is a loss of metabolic flexibility with fatty acid oxidation predominating over glucose oxidation in cardiac mitochondria. As a result, cardiac efficiency is reduced which can impair production of ATP, reduce the pumping capacity of the heart, and impair its ability to tolerate and recover from an ischemic insult.

Existing therapies for treating cardiovascular disease have limitations. Several approaches that focus on restoring blood flow require risky surgical interventions. For example, coronary artery bypass grafting is a major surgery associated with various complications. Treatment of obstructive hypertrophic cardiomyopathy may include septal myectomy, ethanol ablation, or an implantable cardioverter defibrillator, all with associated risks of significant complications.

Many classes of drugs, such as cholesterol-lowering medicine, beta blockers, calcium channel blockers, diuretics, renin-angiotensin-aldosterone system blockade and aldosterone antagonists fail to directly rectify changes in cardiac energy metabolism and/or optimize substrate use for energy production. Those few existing drugs that directly target cardiac substrate energy metabolism have serious adverse effects. The adverse effect profile of these agents has limited their use despite their potential to redress the balance between glucose oxidation and fatty acid oxidation and improve cardiac efficiency. Further, drugs that do not robustly restore glucose oxidation in the heart have limited efficacy because mitigating adverse cardiac remodeling is not addressed. Consequently, existing approaches to improve cardiac function either do not directly target cardiac metabolism or, in the case of those that do alter mitochondrial metabolism, are unsatisfactory. Therefore, no safe, effective therapy exists for millions of people who continue to experience morbidity and die from heart disease each year.

### SUMMARY

The invention relates to compounds, compositions, and methods for treating cardiovascular conditions and increasing the efficiency of cardiac metabolism. Particularly, the invention provides compounds to treat cardiac conditions by improving cardiac mitochondrial metabolism. The compounds shift cardiac metabolism from fatty acid oxidation to glucose oxidation, improving the efficiency of mitochondrial energy generation (i.e., acting as a mitotrope) and thereby the energetic status and function of the heart. The invention provides for compositions and methods of treatment using the compounds. The compounds, compositions, and methods are useful for treating a wide variety of cardiovascular conditions as described herein.

The invention provides compounds for increasing the efficiency of cardiac metabolism. In one aspect, the invention provides a compound represented by formula (X):

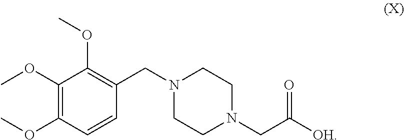 [SMILES: COc1ccc(CN2CCN(CC(=O)O)CC2)c(OC)c1OC]

Such compounds of formula X can be provided in pharmaceutical formulations with pharmaceutically acceptable salts and excipients.

It is believed that the compound of formula X may be further metabolized to compounds of formula (A) or (B):

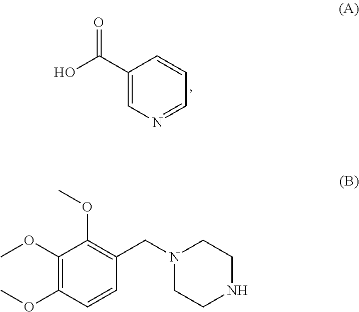 [SMILES: O=C(O)c1cccnc1 ; COc1ccc(CN2CCNCC2)c(OC)c1OC]

Formula (B) is trimetazidine.

In another embodiment, the invention provides compounds of Formula Y, as shown below.

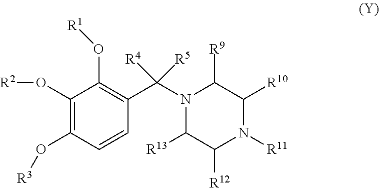

Formula Y comprises one or more substitutions at R¹, R², R³, R⁴, R⁸, R⁹, R¹⁰, R¹¹, R¹², or R¹³, wherein R¹, R², and R³ are independently H or a (C₁-C₄)alkyl group; R⁴ and R⁸ together are ═O, —O(CH₂)ₘO—, or —(CH₂)ₘ—, in which m=2-4, or R⁴ is H and R⁸ is H, OR¹⁴, SR¹⁴, or (CH₂CH₂O)ₙH, in which R¹⁴ is H or a (C₁-C₄)alkyl group and n=1-15; R⁹, R¹⁰, R¹², and R¹³ are independently H or (CH₂CH₂O)_{z}H, in which z=1-6; and R¹¹ comprises a compound that promotes mitochondrial respiration.

In further embodiments, the aryloxy groups independently comprise a methoxy, an ethoxy, an alcohol, an alkoxide, a hydrogen, or a (C₁-C₄)alkyl group. In embodiments, the structure contains 3-22 atoms, not including hydrogen atoms bonded to atoms in ring positions. Relatedly, the structure includes one or more alkyl, alkenyl, or aromatic rings. In embodiments, the structure includes one or more heteroatoms, i.e., atoms other than carbon. For example, the heteroatom may be oxygen, nitrogen, sulfur, or phosphorus.

Such compounds of formula Y can be provided in pharmaceutical formulations with pharmaceutically acceptable salts and excipients.

In another aspect, the invention provides a method of treating cardiac dysfunction in a subject, the method comprising administering to the subject a composition comprising the compound of formula (X) or formula (Y).

The cardiovascular condition is selected from the group consisting of acute coronary syndrome, aneurysm, angina, anthracycline-induced cardiotoxicity, atherosclerosis, cardiac adiposity or steatosis (including that found in conditions such as aortic stenosis, HIV/ART-associated myocardial steatosis, hypertensive heart disease, coronary microvascular dysfunction and generalized lipodystrophy), cardiac ischemia-reperfusion injury, cardiogenic shock, cardiomyopathy (inherited or acquired, including obstructive hypertrophic, non-obstructive hypertrophic, dilated, and restrictive forms), cardiac lipotoxicity, cardioprotection (including during cardiac surgery with cardiopulmonary bypass), cardio-renal syndrome, cerebral vascular disease, chronic coronary syndromes, congenital heart disease, contrast nephropathy, coronary artery disease, coronary heart disease, coronary microvascular dysfunction, diabesity, diabetic cardiomyopathy (including asymptomatic pre-overt heart failure and stage B diabetic cardiomyopathy), heart attack, heart disease, heart failure (all stages and with reduced, mildly reduced or preserved ejection fraction, i.e. including HFrEF, HFmEF and HFpEF), cardiometabolic HFpEF, heart failure after cardiac transplantation including in diabetics, hibernating or stunned myocardium, hypertension, hypertensive heart disease, hypertrophic cardiomyopathy (both non-obstructive and obstructive forms), ischemia with no obstructive coronary artery disease (INOCA), ischemia-reperfusion injury, ischemic heart disease, ischemic cardiomyopathy, lipotoxic cardiomyopathy, metabolic syndrome, microvascular angina, MINOCA, mitochondrial cardiomyopathies, myocardial dysfunction induced by anti-cancer drugs, myocardial infarction, non-ischemic cardiomyopathy, obesity cardiomyopathy, pericardial disease, pericardial (and/or epicardial) fat accumulation, peripheral arterial disease, pulmonary hypertension (PH)—including WHO group 1 (pulmonary arterial hypertension) group 2 (PH due to left heart disease) group 3 (PH due to lung disease) group 4 (chronic thromboembolic PH, CTEPH) and group 5 (PH due to unknown causes)—both primary and secondary, long COVID and post-acute COVID-19 cardiovascular sequelae, rheumatic heart disease, right heart failure, right ventricular failure, stroke, Takotsubo (stress) cardiomyopathy, transient ischemic attack(s), and valvular heart disease (including as medical therapy pre- and/or post-valve repair or replacement), and vasospastic angina. Long COVID and post-acute COVID-19 cardiovascular sequelae is discussed in Raman, 2022, Long COVID: post-acute sequelae of COVID-19 with a cardiovascular focus, Eur Heart J 43, 1157-1172, incorporated by reference herein.

In embodiments, the compounds and compositions may be provided in a dosage form and the dose may be provided by any suitable route or mode of administration. The dose may be provided orally, intravenously, enterally, parenterally, dermally, buccally, topically, transdermally, by injection, subcutaneously, nasally, pulmonarily, or with or on an implantable medical device (e.g., stent or drug-eluting stent or balloon equivalents).

The composition may be provided in one dose per day. The composition may be provided in multiple doses per day. The composition may be provided in two, three, four, five, six, eight, or more doses per day.

The dose or doses may be provided for a defined period. One or more doses may be provided daily for at least one week, at least two weeks, at least three weeks, at least four weeks, at least six weeks, at least eight weeks, at least ten weeks, at least twelve weeks or more.

### DETAILED DESCRIPTION

The invention provides compounds, and methods for administering compositions containing compounds, that improve cardiac mitochondrial bioenergetics, cardiac efficiency and cardiac function to treat cardiovascular conditions.

### Definitions

As used herein, the term “alkoxyl” refers to an alkyl group singularly bonded to an oxygen atom, having the formula R—O. Alkoxyls include, for example, methoxy (CH₃O—) and ethoxy, (CH₃CH₂O—).

A “cycloalkoxyl” refers to a cycloalkyl group singularly bonded to an oxygen atom, which includes “aryloxy” groups, in which an aryl group is singular bonded to oxygen, for example a phenoxy group (C₆H₅O). Similarly, the term “heteroalkoxyl” refers to a heteroalkyl group singularly bonded to an oxygen atom and the term “cycloheteroalkoxyl” refers to a cycloheteroalkyl singularly bonded to an oxygen atom.

As used herein, the term “aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C6-14 aryl”). Unless otherwise specified, each instance of an aryl group is independently optionally substituted, i.e., unsubstituted (an “unsubstituted aryl”) or substituted (a “substituted aryl”) with one or more substituents.

As used herein, the term “alkyl” refers to a saturated monovalent chain of carbon atoms, which may be optionally branched, the term “alkenyl” refers to an unsaturated monovalent chain of carbon atoms including at least one double bond, which may be optionally branched, the term “alkylene” refers to a saturated bivalent chain of carbon atoms, which may be optionally branched, and the term “cycloalkylene” refers to a saturated bivalent chain of carbon atoms, which may be optionally branched, a portion of which forms a ring.

As used herein, the term “heterocycle” refers to a chain of carbon and heteroatoms, wherein the heteroatoms are selected from nitrogen, oxygen, and sulfur, at least a portion of which, including at least one heteroatom, form a ring, such as, but not limited to, tetrahydrofuran, aziridine, pyrrolidine, oxazolidine, 3-methoxypyrrolidine, 3-methylpiperazine, and the like. As used herein, the term “acyl” refers to hydrogen, alkyl, cycloalkyl, alkenyl, cycloalkenyl, heterocyclyl, optionally substituted aryl, optionally substituted arylalkyl, optionally substituted heteroaryl, and optionally substituted heteroarylalkyl attached as a substituent through a carbonyl group.

As used herein, the term “alkylene” generally refers to a bivalent saturated hydrocarbon group wherein the hydrocarbon group may be a straight-chained or a branched-chain hydrocarbon group.

The term “cycloalkyl” as used herein generally refers to a monovalent chain of carbon atoms, at least a portion of which forms a ring. The term “cycloalkenyl” as used herein refers to a monovalent chain of carbon atoms containing one or more unsaturated bonds, at least a portion of which forms a ring.

As used herein, the term “cycloheteroalkyl” generally refers to an optionally branched chain of atoms that includes both carbon and at least one heteroatom, where the chain optionally includes one or more unsaturated bonds, and where at least a portion of the chain forms one or more rings. As used herein, it is understood that the term “cycloheteroalkyl” also includes “heterocycloalkyl,” “heterocycle,” and “heterocyclyl.” The term “heterocycloalkenyl” as used herein refers to a monovalent chain of carbon atoms and heteroatoms containing one or more unsaturated bonds, a portion of which forms a ring, wherein the heteroatoms are selected from nitrogen, oxygen or sulfur. Illustrative cycloheteroalkyls include, but are not limited to, tetrahydrofuryl, bis(tetrahydrofuranyl), pyrrolidinyl, tetrahydropyranyl, piperidinyl, morpholinyl, piperazinyl, homopiperazinyl, quinuclidinyl, dihydrofuryl, pyrrollinyl, dihydropyranyl, and the like. It is also to be understood that cycloheteroalkyl includes polycyclic radicals, including fused bicycles, spiro bicycles, and the like.

“Heteroaryl” refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system.

“Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. “Heteroaryl” refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system.

As used herein, “haloalkyl” is generally taken to mean an alkyl group wherein one or more hydrogen atoms is replaced with a halogen atom, independently selected in each instance from the group consisting of fluorine, chlorine, bromine and iodine.

“Halo” or “halogen,” independently or as part of another substituent, generally refers to a fluorine (F), chlorine (Cl), bromine (Br), or iodine (I) atom. The term “halide” by itself or as part of another substituent, refers to a fluoride, chloride, bromide, or iodide atom.

As used herein, the term “optionally substituted” includes a wide variety of groups that replace one or more hydrogens on a carbon, nitrogen, oxygen, or sulfur atom, including monovalent and divalent groups. For example, optional substitution of carbon includes, but is not limited to, halo, hydroxy, alkyl, alkoxy, haloalkyl, haloalkoxy, aryl, arylalkyl, acyl, acyloxy, and the like. In one aspect, optional substitution of aryl carbon includes, but is not limited to, halo, amino, hydroxy, alkyl, alkenyl, alkoxy, arylalkyl, arylalkyloxy, hydroxyalkyl, hydroxyalkenyl, alkylene dioxy, aminoalkyl, where the amino group may also be substituted with one or two alkyl groups, arylalkyl groups, and/or acyl groups, nitro, acyl and derivatives thereof such as oximes, hydrazones, and the like, cyano, alkylsulfonyl, alkylsulfonylamino, and the like. Illustratively, optional substitution of nitrogen, oxygen, and sulfur includes, but is not limited to, alkyl, haloalkyl, aryl, arylalkyl, acyl, and the like, as well as protecting groups, such as alkyl, ether, ester, and acyl protecting groups, and pro-drug groups. It is further understood that each of the foregoing optional substituents may themselves be additionally optionally substituted, such as with halo, hydroxy, alkyl, alkoxy, haloalkyl, haloalkoxy, and the like.

It is understood that substitutions and any functional group may be independently ortho-, para-, or meta-. It is understood that cyclic groups may be aromatic or non-aromatic.

“Stereoisomers”: It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of x electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic and may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. (See, e.g., Berge, et al., J. Pharm. Sci. (1977) 66(1): 1-79, the entirety of the contents of which are incorporated by reference herein).

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

The invention provides compounds for increasing the efficiency of cardiac metabolism and increasing cardiac energetics. In one aspect, the invention provides a compound represented by Formula (X) or (Y), and compositions including such compounds. The compositions may be provided in pharmaceutical formulations with pharmaceutically acceptable salts and excipients.

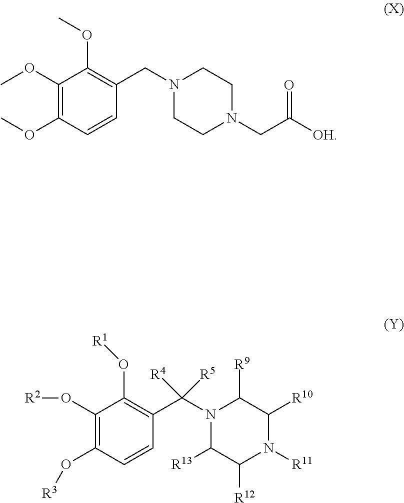

Formula Y may comprise one or more substitutions at R¹, R², R³, R⁴, R⁸, R⁹, R¹⁰, R¹¹, R¹², or R¹³, wherein R¹, R², and R³ are independently H or a (C₁-C₄)alkyl group; R⁴ and R⁸ together are ═O, —O(CH₂)ₘO—, or —(CH₂)ₘ—, in which m=2-4, or R⁴ is H and R⁸ is H, OR¹⁴, SR¹⁴, or (CH₂CH₂O)ₙH, in which R¹⁴ is H or a (C₁-C₄)alkyl group and n=1-15; R⁹, R¹⁰, R¹², and R¹³ are independently H or (CH₂CH₂O)_{z}H, in which z=1-6; and R¹¹ comprises a compound that promotes mitochondrial respiration.

The aryloxy groups may independently comprise a methoxy, an ethoxy, an alcohol, an alkoxide, a hydrogen, or a (C₁-C₄)alkyl group. In embodiments, the structure may contain 3-22 atoms, not including hydrogen atoms bonded to atoms in ring positions. Relatedly, the structure may include one or more alkyl, alkenyl, or aromatic rings. In other embodiments, the structure may include one or more heteroatoms, i.e., atoms other than carbon. For example, the heteroatom may be oxygen, nitrogen, sulfur, or phosphorus.

### Cardiac Conditions

In coronary artery disease (CAD), blood flow to the heart muscle is reduced, usually due to accumulation of atherosclerotic plaque in the epicardial coronary arteries. This may present as chronic (stable) disease, for example, exertional angina, or as acute (unstable) disease, for example, acute coronary syndrome with myocardial infarction. Other manifestations of atherosclerotic cardiovascular disease include peripheral arterial disease and stroke. As discussed in more detail below, in some patients, CAD can lead to heart failure (sometimes termed ‘ischemic cardiomyopathy’)—a form of heart failure with reduced ejection fraction (HFrEF)—resulting from irreversible loss of a significant amount of myocardial tissue due to myocardial infarction and/or reversible loss of contractility resulting from chronically ischemic but viable myocardium. As discussed in Kloner, 2020, Stunned and hibernating myocardium: Where are we nearly 4 decades later?, J Am Heart Assoc 9(3):1-11, incorporated by reference herein, the viable myocardium may improve following revascularization in which case it is referred to as hibernating myocardium.

As noted above, the heart is dependent on a continuous energy supply in the form of adenosine triphosphate (ATP) for contractile function. The heart has the highest energy demands, measured as ATP per gram of tissue, of any organ, with a complete turnover of its ATP pool every ˜10 seconds. Energy demands and heart failure are discussed in detail in Neubauer, 2007, The failing heart—an engine out of fuel, N Engl J Med 356(11):1140-1151, and Ingwall, 2004, Is the failing heart energy starved? On using chemical energy to support cardiac function, Circ Res 95(2):135-145, both of which are incorporated by reference herein. The amount of energy required to support contractile function including excitation-contraction coupling and diastolic active relaxation (a highly energy-demanding process involving a rapid reduction in ventricular pressure i.e., diastolic function), basal metabolic processes, and maintenance of ionic homeostasis is almost exclusively met by oxidative phosphorylation of substrates in the mitochondria. In cardiac disease states, a mismatch between ATP generation and demand leading to energy deprivation occurs, and is exacerbated further in the context of stress.

Given the heart's unrelenting high energy requirements to meet the perpetual demands of contractility, reduced efficiency of energy generation by the heart and the ensuing energy deficit has profound adverse consequences. As a corollary, energetic impairment (which can be measured non-invasively by the phosphocreatine/ATP ratio using ³¹P-magnetic resonance spectroscopy) is a major feature and contributor to most forms of heart disease.

Decreased cardiac efficiency occurs in pathophysiological disease states such as heart failure, ischemic heart disease, inherited cardiomyopathy (e.g., HCM), and diabetic heart disease. Archetypal examples include diabetic cardiomyopathy and obesity cardiomyopathy. In part, this is due to an increased reliance on fatty acid oxidation over glucose oxidation. Glucose oxidation is a more efficient pathway for energy production, as measured by the number of ATP molecules produced per O₂ molecule consumed (P/O˜2.58), than is fatty acid oxidation (P/O˜2.3) and that of the ketone body β-hydroxybutyrate (P/O˜2.5). The importance of this is highlighted by the observation that the heart utilizes more oxygen/gram of tissue than any other organ. As is described in Knaapen, 2007, Myocardial energetics and efficiency: current status of the noninvasive approach, Circ 115(7):918-927, incorporated by reference herein, decreased cardiac efficiency arises substantially from changes in mitochondrial energy metabolism, and is thought to contribute to the progression of the disease. Decreased cardiac efficiency may reflect the ratio between useful cardiac work to myocardial oxygen consumption (MVO₂).

Mitochondria are sub-cellular organelles in which metabolites derived from carbon substrates such as glucose and fatty acids are oxidized to produce high-energy molecules. Under physiological conditions, the healthy heart is metabolically flexible and with respect to substrate utilization, able to dynamically use a range of carbon substrates to generate ATP, primarily of free fatty acids (FFA) and, glucose, as well as contributions from lactate, ketone bodies, and several amino acids to ensure close coupling of oxidative phosphorylation with the rate of ATP hydrolysis across a range of states such as feeding, fasting and exercise.

Glucose oxidation and fatty acid oxidation are energy-producing metabolic pathways that compete with each other for substrates. In glucose oxidation, glucose is broken down to pyruvate via glycolysis in the cytosol of the cell. Pyruvate then enters the mitochondria, where it is converted to acetyl coenzyme A (acetyl-CoA). In beta-oxidation of fatty acids, which occurs in the mitochondria, two-carbon units from long-chain fatty acids are sequentially converted to acetyl-CoA.

The remaining steps in energy production from glucose oxidation of glucose and fatty acid oxidation are common to the two pathways. Acetyl-CoA is oxidized to carbon dioxide (CO₂) via the citric acid cycle, which results in the conversion of nicotinamide adenine dinucleotide (NAD⁺) to its reduced form, NADH, as well as generation of FADH₂. Both NADH and FADH₂, in turn, donate electrons to drive the mitochondrial electron transport chain. The electron transport chain comprises a series of four mitochondrial membrane-bound complexes that transfer electrons via redox reactions. In doing so, the complexes pump protons across the membrane to create a proton gradient. The redox reactions of the electron transport chain require molecular oxygen (O₂) as the final electron acceptor. In the final step of mitochondrial energy production, the proton gradient enables another membrane-bound enzymatic complex (Complex V or ATP synthase) to form high-energy ATP molecules from ADP, which are the source of energy for most cellular reactions.

In certain types of heart disease, such as heart failure (including HFrEF and HFpEF), type 2 diabetes, obesity, and inherited cardiomyopathy (e.g., HCM), there is profound cardiac metabolic remodeling and metabolic inflexibility, with fatty acid oxidation predominating over glucose oxidation in cardiac mitochondria. As a result, cardiac efficiency is reduced which can impair cardiac energetics (i.e., production of ATP), reduce the pumping capacity of the heart (including both systolic, i.e., contractile, and diastolic, i.e., filling, phases) and impair its ability to tolerate and recover from an ischemic insult. For example, in type 2 diabetes and/or obesity there is a near-exclusive overreliance of the myocardium on free fatty acids for energy generation, impaired cardiac glucose uptake (reflecting cardiac insulin resistance), and a compromised ability to switch fuel utilization which have important adverse consequences.

Increasing fatty acid (FA) oxidation in the heart reciprocally decreases glucose oxidation, and vice versa (known as the Randle cycle) and leads to a higher myocardial volume oxygen (MVO₂) relative to hearts predominantly metabolizing glucose. Increased FA oxidation increases delivery of reducing equivalents (NADH and FADH₂) to the mitochondrial electron transport chain, but at the same time also adversely increases generation of reactive oxygen species such as superoxides, as well as lipid peroxides which activate mitochondrial uncoupling resulting in proton leak across the inner mitochondrial membrane, uncoupling oxidative phosphorylation from ATP production. Increased FA utilization also results in futile cycling of FA intermediates and of FAs in and out of the intracellular triacylglycerol pool resulting in greater ATP consumption for non-contractile purposes.

Glucose oxidation is a more efficient source of energy generation than fatty acids and other substrates (including ketone bodies), i.e., glucose oxidation produces more ATP for the same amount of oxygen available, reflecting its higher phosphate/oxygen ratio (P/O ratio, reflecting the number of ATP molecules produced per atom of oxygen). While cardiac efficiency calculated solely stoichiometrically on the basis of the P/O ratio indicates an increase in efficiency from switching from fatty acid to glucose-based metabolism of ˜10-12%, the substantial energy wasting effects of fatty acid metabolism outlined above (mitochondrial uncoupling, futile energy cycles) result in a much greater impairment in cardiac efficiency, ranging from ˜25-50%. These energy wasting effects are described in detail in Korvald, 2000, Myocardial substrate metabolism influences left ventricular energetics in vivo, Am J Physiol Heart Circ Physiol 278(4):1345-1351. Accordingly, there is a marked energetic and mechanical efficiency advantage to shifting cardiac metabolism towards glucose oxidation.

Other metabolic changes contribute to decreased cardiac efficiency in patients with heart disease. For example, overall mitochondrial oxidative metabolism can be impaired in heart failure, and energy production is decreased in ischemic heart disease due to a limited supply of oxygen at rest or under conditions of increased myocardial oxygen demand, such as exercise inducing ischemia. As a corollary, stimulation of myocardial glucose oxidation will improve post-ischemic recovery and cardiac efficiency following a period of ischemia and reperfusion. In addition to the reduction in mitochondrial oxidative capacity, the failing heart is characterized by increased glycolysis uncoupled from glucose oxidation, reducing energy production (2 compared with 31 ATP molecules per glucose molecule if the pyruvate from glycolysis is oxidized) and generating lactate which leads to intracellular H″ accumulation impairing cellular function and intracellular Ca²⁺ homeostasis.

Highlighting the importance of myocardial efficiency as a therapeutic target, treatments that enhance mechanical efficiency of the heart are associated with beneficial outcomes. For example, the use of the beta blocker metoprolol or cardiac resynchronization therapy in heart failure, as described in Beanlands, 2000, The effects of beta(1)-blockade on oxidative metabolism and the metabolic cost of ventricular work in patients with left ventricular dysfunction: A double-blind, placebo-controlled, positron-emission tomography study, Circ 102(17):2070-2075, and Ukkonen, 2003, Effect of cardiac resynchronization on myocardial efficiency and regional oxidative metabolism, Circ 107(1):28-31, both of which are incorporated by reference herein.

However, previous classes of drugs, such as cholesterol-lowering medicine, beta blockers, calcium channel blockers, diuretics, renin-angiotensin-aldosterone system (RAAS) blockade and aldosterone antagonists fail to directly rectify changes in cardiac energy metabolism and/or optimize substrate use for energy production. For example, most treatments for angina due to coronary artery disease (including coronary artery bypass grafting, percutaneous coronary intervention, pharmacological approaches) aim to reduce myocardial oxygen consumption (MVO₂) or increase oxygen delivery through one or more of increased coronary blood flow, reduction of heart rate and/or reduced cardiac contractility. Such approaches are frequently limited by hemodynamic side effects (excessive fall in blood pressure or heart rate resulting in symptoms such as light-headedness or syncope) and do not address the underlying cardiac mechanical efficiency and/or inappropriate or excessive reliance on FA oxidation associated with inefficient energy generation.

Those few existing drugs that directly target cardiac substrate energy metabolism have serious adverse effects, for example, high levels of transaminitis with etomoxir, peripheral neuropathy with dichloroacetate, and hepatotoxicity and peripheral neuropathy with perhexiline. The adverse effect profile of these agents has limited their use.

Compounds of the invention increase the efficiency of cardiac metabolism and increase cardiac energetics without serious side effects. Compositions that shift cardiac metabolism from fatty acid oxidation to glucose oxidation can be classified based on their mechanism of action. See Fillmore, N., et al., Mitochondrial fatty acid oxidation alterations in heart failure, ischemic heart disease and diabetic cardiomyopathy, Brit. J. Pharmacol. 171:2080-2090 (2014), the contents of which are incorporated herein by reference. One class of glucose-shifting compounds includes compounds that inhibit fatty acid oxidation directly. Compounds in this class include inhibitors of malonyl CoA decarboxylase (MCD), carnitine palmitoyl transferase 1 (CPT-1), or mitochondrial fatty acid oxidation. Mitochondrial fatty acid oxidation inhibitors include trimetazidine and other compounds described in International Patent Publication No. WO 2002/064576, the contents of which are incorporated herein by reference. Trimetazidine binds to distinct sites on the inner and outer mitochondrial membranes and affects both ion permeability and metabolic function of mitochondria. Morin, D., et al., Evidence for the existence of [³H]-trimetazidine binding sites involved in the regulation of the mitochondrial permeability transition pore, Brit. J. Pharmacol. 123:1385-1394 (1998), the contents of which are incorporated herein by reference. MCD inhibitors include CBM-301106, CBM-300864, CBM-301940, 5-(1,1,1,3,3,3-hexafluoro-2-hydroxypropan-2-yl)-4,5-dihydroisoxazole-3-carboxamides, methyl 5-(N-(4-(1,1,1,3,3,3-hexafluoro-2-hydroxypropan-2-yl)phenyl) morpholine-4-carboxamido) pentanoate, and other compounds described in Chung, J. F., et al., Discovery of Potent and Orally Available Malonyl-CoA Decarboxylase Inhibitors as Cardioprotective Agents, J. Med. Chem. 49:4055-4058 (2006); Cheng J. F. et al., Synthesis and structure-activity relationship of small-molecule malonyl coenzyme A decarboxylase inhibitors, J. Med. Chem. 49:1517-1525 (2006); U.S. Patent Publication No. 2004/0082564; and International Patent Publication No. WO 2002/058698, the contents of each of which are incorporated herein by reference. CPT-1 inhibitors include oxfenicine, perhexiline, etomoxir, and other compounds described in International Patent Publication Nos. WO 2015/018660; WO 2008/109991; WO 2009/015485; and WO 2009/156479; and U.S. Patent Publication No. 2011/0212072, the contents of each of which are incorporated herein by reference.

Heart failure (HF) is a clinical syndrome due to a structural and/or functional abnormality of the heart that results in elevated intra-cardiac pressures and/or inadequate cardiac output at rest and/or during exercise. Clinically, heart failure is characterized by symptoms such as breathlessness, peripheral edema, exercise intolerance and fatigue, which may be accompanied by signs such as tachycardia, elevated jugular venous pressure, pulmonary crackles and/or peripheral edema. Most heart failure arises due to (systolic and/or diastolic) myocardial dysfunction, but heart failure can also be primarily a result of abnormalities of the endocardium, pericardium, valves, heart rhythm and/or cardiac conduction. Heart failure is traditionally categorized into distinct phenotypes based on a measurement of left ventricular ejection fraction (LVEF), although with a realization in the field that LVEF is a continuum, and that heart failure spans the full spectrum of L VEF.

Heart failure can be grouped into main categories of: HF with reduced ejection fraction (HFrEF, with LVEF≤40%); heart failure with mildly reduced ejection fraction (HFmEF, with LVEF 41-49%) and HFimpEF (HF with improved EF) reflecting previous LVEF in the HFrEF range (≤40%) and a follow-up measurement of >40%; and HF with preserved ejection fraction (HFpEF, with LVEF≥50%).

Heart failure is discussed in detail in McDonagh, 2021, ESC guidelines for the diagnosis and treatment of acute and chronic heart failure, Eur Heart Journal 42:3599-3726, and Heidenreich, 2022, 2022 AHA/ACC/HFSA Guideline for the management of heart failure: Executive summary: A report of the American College of Cardiology/American Heart Association joint committee on clinical practice guidelines, Journal Am Col Card 79, 17:1757-1780, both of which are incorporated by reference herein. The main categories of heart failure are described in Diagnostic criteria for all categories require concomitant symptoms (=signs) of heart failure.

Heart failure is divided by the American College of Cardiology (ACC) and the American Heart Association (AHA) into stages A to D, reflecting the development and progression of heart failure from asymptomatic (stages A and B) through to symptomatic stages (stages C and D). Stage A (at risk for HF) refers to patients at risk for heart failure but without symptoms, structural heart disease, or cardiac biomarkers of stretch or injury (e.g., patients with hypertension, atherosclerotic CVD, diabetes, metabolic syndrome and obesity, exposure to cardiotoxic agents, genetic variant for cardiomyopathy, or positive family history of cardiomyopathy). Stage B (pre-HF) refers to those without current or previous symptoms/signs of HF but with evidence of 1 of: structural heart disease; evidence for increased filling pressures; risk factors and increased natriuretic peptide levels or persistently raised cardiac troponin in the absence of competing diagnoses. Stage C (symptomatic HF) refers to those with structural heart disease with current or previous symptoms of HF, whilst stage D (advanced HF) refers to those with marked HF symptoms that interfere with daily life and who experience recurrent hospitalizations despite attempts to optimize guideline-directed medical therapy (GDMT).

The clinical presentation of heart failure may be chronic or acute, with chronic referring to a gradual, progressive onset of symptoms. Patients with chronic heart failure may experience a deterioration termed decompensated HF which may be rapid (i.e., acute) or chronic in onset and require hospitalization and/or parenteral (intravenous) diuretic treatment. Patients with symptomatic Stage C HF can also be classified based on the trajectory of their symptoms as: new onset/de novo HF; resolution of symptoms; persistent HF; worsening HF.

The etiology of HF is broad and includes: CAD, hypertension, familial or genetic cardiomyopathies (e.g. dilated, hypertrophic, restrictive), peri-partum cardiomyopathy, stress cardiomyopathy (Takotsubo syndrome), toxin- or substance abuse-induced (such as alcohol, cocaine, methamphetamine, iron or copper overload), congenital heart disease, drug-induced (e.g. chemotherapy such as anthracyclines, trastuzumab, immune checkpoint blockade, proteasome inhibitors, VEGF inhibitors), infiltrative disorders (e.g. amyloid, sarcoid, neoplastic), storage diseases (e.g. hemochromatosis, Fabry disease, glycogen storage diseases), endomyocardial disease (e.g. carcinoid, endomyocardial fibrosis, radiotherapy), metabolic disorders (e.g. diabetes, obesity, thiamine or selenium deficiency), endocrine disease (e.g. thyroid disease, acromegaly, pheochromocytoma), myocarditis (infectious, toxin or medication, immunological, hypersensitivity), rheumatological and autoimmune disease, neuromuscular disease (e.g. Friedreich's ataxia, muscular dystrophy), heart rhythm-related (e.g. tachycardia-induced cardiomyopathy, right ventricular pacing, recurrent premature ventricular complexes), valvular heart disease (e.g. primary valve disease such as aortic stenosis or organic mitral regurgitation, secondary valve disease such as functional mitral regurgitation, congenital valve disease), infective (e.g., viral myocarditis, HIV, Chagas disease, Lyme disease), pericardial disease (e.g., calcification or infiltrative disease leading to pericardial constriction).

HFpEF is defined hemodynamically as a clinical syndrome in which the heart is unable to pump blood adequately to meet metabolic demands at normal cardiac filling pressures. For example, as described in Borlaug, 2020, Evaluation and management of heart failure with preserved ejection fraction, Nat Rev Cardiol, 17(9):559-573, incorporated by reference herein, invasive exercise testing allows a direct assessment of the parameters that define HFpEF. Specifically, HFpEF is defined invasively by an elevated pulmonary capillary wedge pressure (PCWP)≥15 mmHg at rest and/or ≥25 mmHg during exercise. Highlighting the value of invasive exercise testing, for example, as described in Borlaug, 2010, Exercise hemodynamics enhance diagnosis of early heart failure with preserved ejection fraction, Circ Heart Fail 3(5):588-595, incorporated by reference herein, a substantial proportion of patients with HFpEF who may be euvolemic at rest with normal natriuretic peptides develop abnormalities in left-sided heart filling pressures only on exertion. This population of patients is likely to represent an earlier stage of HFpEF in whom intermittent excessive rises in filling pressures and wall stress present during exercise are not evident at rest; as a corollary non-invasive measures of markers of congestion may be normal or near-normal in clinic in the resting state. Importantly, elevated left ventricular filling pressures (even when only restricted to exercise) identifies patients at increased risk for HF hospitalization or mortality, as described in Dorfs, 2014, Pulmonary capillary wedge pressure during exercise and long-term mortality in patients with suspected heart failure with preserved ejection fraction, Eur Heart 21; 35(44):3103-3112, and Eisman, 2018, Pulmonary capillary wedge pressure patterns during exercise predict exercise capacity and incident heart failure, Circ Heart Fail 11(5):1-19, both of which are incorporated by reference herein.

The cardinal symptom of HFpEF is exercise intolerance manifest as exertional dyspnea and/or fatigue. This results in severely impaired exercise capacity with marked functional impairment, reduced quality of life and is associated with increased mortality. Exercise capacity in HFpEF is undermined by multiple central and peripheral defects in the pathway of oxygen transport and utilization, including reduced cardiac output and an impaired peripheral response to exercise, specifically impaired skeletal muscle diffusion capacity, and/or a reduced aerobic capacity of skeletal muscle. Many patients harbor a combination of defects, as described in Houstis, 2017, Exercise intolerance in heart failure with preserved ejection fraction: Diagnosing and ranking its causes using personalized O2 pathway analysis, Circ 137(2):148-161, incorporated by reference herein.

A key determinant of exercise intolerance in HFpEF is reduced cardiovascular reserve, as described in Pfeffer, 2019, Heart failure with preserved ejection fraction in perspective, Circ Res 124(11):1598-1617, incorporated by reference herein. This may include impaired LV filling/diastolic dysfunction (lusitropy), impaired LV contractility (inotropy) and ventricular systolic reserve, chronotropic incompetence (chronotropy), left atrial dysfunction and remodeling, right heart abnormalities (increased right-sided filling pressures, reduced RV function and contractile reserve, pulmonary hypertension and pulmonary vascular remodeling), with associated enhanced diastolic ventricular interdependence, abnormal ventricular-vascular coupling with central arterial stiffening and impaired arterial compliance, resistance and elastance reserve, as described in Reddy, 2017, Arterial stiffening with exercise in patients with heart failure and preserved ejection fraction, J Am Coll Cardiol 70(2):136-148, incorporated by reference herein.

In a proportion of patients, in addition to limitations in cardiac output reserve, abnormalities in peripheral oxygen extraction and utilization reduce peak oxygen consumption. These abnormalities include reduced vasodilatory reserve, endothelial dysfunction, capillary rarefaction, excess muscle fat infiltration (myosteatosis), reduced skeletal muscle mitochondrial density, bioenergetics and function. As described in Pandey, 2021, exercise intolerance in older adults with heart failure with preserved ejection fraction: JACC state-of-the-art review, J Am Coll Cardiol 78(11)1166-1187, incorporated by reference herein, the effects of these abnormalities may be compounded by reduced skeletal muscle mass common in elderly patients with HFpEF.

Conditions that can lead to heart failure include inherited diseases of the heart muscle such as hypertrophic cardiomyopathy (HCM) in which the muscular wall of the main pumping chamber of the heart (the left ventricle) becomes abnormally thickened in the absence of loading conditions, associated with impaired filling of the heart (diastolic dysfunction) and myocardial ischemia. HCM is subdivided into obstructive (where there is resting or provokable/inducible left ventricular outflow tract obstruction impairing the blood flow out of the heart and associated leak of the mitral valve [mitral regurgitation] or non-obstructive forms. Both are associated with exertional dyspnea, reduced functional capacity, supraventricular (particularly atrial fibrillation) and ventricular arrhythmias, thromboembolic events (such as stroke), myocardial fibrosis, progressive heart failure (including advanced or end-stage which may be associated with ventricular dilatation, wall thinning and features of HFrEF), and increased risk of sudden cardiac death usually due to malignant ventricular arrhythmia.

Conditions such as diabetes or pre-diabetes, obesity, the metabolic syndrome, hypertension and dyslipidemia increase the risk of coronary artery disease (CAD) and heart failure, and cardiomyopathy. Diabetes mellitus (both type 1 and type 2) can also lead to myocardial dysfunction, structural abnormalities and symptomatic heart failure even in the absence of other major driving factors such as significant epicardial CAD, hypertension or valvular heart disease—termed diabetic cardiomyopathy. In asymptomatic patients this represents a form of stage B HF. Characteristic abnormalities may include, for example, increased LV mass and wall thickness (i.e. LV hypertrophy), left atrial (LA) enlargement, LV diastolic dysfunction and impaired global longitudinal strain (GLS) the latter a marker of systolic dysfunction, and in some individuals an elevation in natriuretic peptides (e.g. NT-proBNP), as described in Stanton, 2021, Asymptomatic diabetic cardiomyopathy: an underrecognized entity in type 2 diabetes, Curr Diab Rep 21(10):41, incorporated by reference herein.

Other hallmark features of the diabetic heart, even in asymptomatic subjects, are discussed in detail in Levelt, 2016, Ectopic and visceral fat deposition in lean and obese patients with type 2 diabetes, J Am Coll Cardiol 68(1):53-63, and Rider, 2020, Noninvasive in vivo assessment of cardiac metabolism in the healthy and diabetic human heart using hyperpolarized ¹³C MRI, Circ Res 126(6):725-736, both of which are incorporated by reference herein. For example, features of the diabetic heart may include impaired energetics, which may be measured as the phosphocreatine/adenosine triphosphate concentration ratio [PCr/ATP], thus reflecting the available energy reserve in the heart, myocardial steatosis with or without obesity, and reduced flux through pyruvate dehydrogenase (PDH). PDH catalyzes the oxidative decarboxylation of pyruvate to form acetyl-CoA and plays a pivotal role linking glycolysis to the oxidative pathway of the citric acid cycle.

Highlighting the importance of myocardial metabolic and energetic dysregulation, energetic deficit is present in the diabetic heart even before changes in systolic function, for example, LV ejection fraction (LVEF) or increased LV mass, as described in Levelt, 2016, Cardiac energetics, oxygenation, and perfusion during increased workload in patients with type 2 diabetes mellitus, Eur Heart 37(46):3461-3469, incorporated by reference herein. Energetic deficit may be identified non-invasively as reduced resting PCr/ATP measured by ³¹P-MRS, with further reduction during exercise. Recently abnormal LV diastolic responses to exercise have also been identified in individuals with type 2 diabetes without HF, including blunting of peak diastolic filling rate and abnormal dilatation of the LA and RA during exercise.

Impaired energetic reserve has also been identified in patients with overt HFpEF, with a gradient of progression in energetic deficit across the disease spectrum illustrated by the relative cardiac energetic levels in asymptomatic patients with type 2 diabetes, symptomatic HFpEF, to an advanced form of HFpEF physiology with restriction due to amyloid cardiomyopathy. Impaired energetic reserve is further discussed in Burrage, 2021, Energetic basis for exercise-induced pulmonary congestion in heart failure with preserved ejection fraction, Circ 144(21):1664-1678, and Phan, 2009, Heart failure with preserved ejection fraction is characterized by dynamic impairment of active relaxation and contraction of the left ventricle on exercise and associated myocardial energy deficiency, J Am Coll Cardiol 54(5):402-409, both of which are incorporated by reference herein.

As noted above, the heart has an absolute requirement for chemical energy in the form of ATP to support normal systolic and diastolic performance, including for key ATP-utilizing reactions such as force production by the heart (dependent on ATP hydrolysis by the myosin motor, equating to ≈400 ATP molecules consumed per half-sarcomere thick filament), the Ca²⁺-ATPase in the sarcoplasmic reticulum, and the sarcolemmal Na⁺—K⁺-ATPase. Additionally, diastolic active relaxation involves a rapid reduction in ventricular pressure and is highly energy-demanding, requiring ATP for dissociation and reuptake of Ca²⁺ from thin filament-associated troponin C into the sarcoplasmic reticulum.

As a corollary, excessively slow relaxation, particularly during higher heart rates such as that associated with exercise, can elevate early and late diastolic filling pressures, and via the Starling mechanism, reduce stroke volume. The mechanisms of diastolic dysfunction are detailed in Borlaug, 2006, Mechanisms of diastolic dysfunction in heart failure, Trends Card Med 16(8):276-279, incorporated by reference herein. Indeed, patients with HFpEF exhibit a dynamic impairment of LV active relaxation as well as LV contractile performance on exercise with a striking prolongation in time to peak LV filling normalized for heart rate during exercise compared to shortening in normal controls (the latter consistent with normal physiological enhanced LV active relaxation), highlighting the consequences of exercise challenge in the face of reduced resting myocardial energetic reserves. Recently abnormal LV diastolic responses to exercise have also been identified in individuals with type 2 diabetes without HF, including blunting of peak diastolic filling rate and abnormal dilatation of the left atrial (LA) and right atrial (RA) during exercise.

In another aspect, the invention provides a method of treating cardiac dysfunction in a subject, the method comprising administering to the subject a composition comprising the compound of formula (X) or (Y). The cardiovascular condition may be selected from the group consisting of acute coronary syndrome, aneurysm, angina, anthracycline-induced cardiotoxicity, atherosclerosis, cardiac adiposity or steatosis (including that found in conditions such as aortic stenosis, HIV/ART-associated myocardial steatosis, hypertensive heart disease, coronary microvascular dysfunction and generalized lipodystrophy), cardiac ischemia-reperfusion injury, cardiogenic shock, cardiomyopathy (inherited or acquired, including obstructive hypertrophic, non-obstructive hypertrophic, dilated, and restrictive forms), cardiac lipotoxicity, cardioprotection (including during cardiac surgery with cardiopulmonary bypass), cardio-renal syndrome, cerebral vascular disease, chronic coronary syndromes, congenital heart disease, contrast nephropathy, coronary artery disease, coronary heart disease, coronary microvascular dysfunction, diabesity, diabetic cardiomyopathy (including asymptomatic pre-overt heart failure and stage B diabetic cardiomyopathy), heart attack, heart disease, heart failure (all stages and with reduced, mildly reduced or preserved ejection fraction, i.e. including HFrEF, HFmEF and HFpEF), cardiometabolic HFpEF, heart failure after cardiac transplantation including in diabetics, hibernating or stunned myocardium, hypertension, hypertensive heart disease, hypertrophic cardiomyopathy (both non-obstructive and obstructive forms), ischemia with no obstructive coronary artery disease (INOCA), ischemia-reperfusion injury, ischemic heart disease, ischemic cardiomyopathy, lipotoxic cardiomyopathy, metabolic syndrome, microvascular angina, MINOCA, mitochondrial cardiomyopathies, myocardial dysfunction induced by anti-cancer drugs, myocardial infarction, non-ischemic cardiomyopathy, obesity cardiomyopathy, pericardial disease, pericardial (and/or epicardial) fat accumulation, peripheral arterial disease, pulmonary hypertension (PH)—including WHO group 1 (pulmonary arterial hypertension) group 2 (PH due to left heart disease) group 3 (PH due to lung disease) group 4 (chronic thromboembolic PH, CTEPH) and group 5 (PH due to unknown causes)—both primary and secondary, long COVID and post-acute COVID-19 cardiovascular sequelae, rheumatic heart disease, right heart failure, right ventricular failure, stroke, Takotsubo (stress) cardiomyopathy, transient ischemic attack(s), and valvular heart disease (including as medical therapy pre- and/or post-valve repair or replacement), and vasospastic angina.

Myocardial steatosis and its relationship to cardiac dysfunction in aortic stenosis is discussed in Mahmod M, Bull S, Suttie J J, Pal N, Holloway C, Dass S, Myerson S G, Schneider J E, De Silva R, Petrou M, Sayeed R, Westaby S, Clelland C, Francis J M, Ashrafian H, Karamitsos T D, Neubauer S. Myocardial steatosis and left ventricular contractile dysfunction in patients with severe aortic stenosis. Circ Cardiovasc Imaging. 2013 September; 6(5):808-16. doi: 10.1161/circimaging.113.000559. Epub 2013 Jul. 5. PMID: 23833283, the entirety of the contents of which are incorporated by reference herein.

HIV/ART-associated myocardial steatosis is discussed in Neilan T G, Nguyen K L, Zaha V G, Chew K W, Morrison L, Ntusi N A B, Toribio M, Awadalla M, Drobni Z D, Nelson M D, Burdo T H, Van Schalkwyk M, Sax P E, Skiest D J, Tashima K, Landovitz R J, Daar E, Wurcel A G, Robbins G K, Bolan R K, Fitch K V, Currier J S, Bloomfield G S, Desvigne-Nickens P, Douglas P S, Hoffmann U, Grinspoon S K, Ribaudo H, Dawson R, Goetz M B, Jain M K, Warner A, Szczepaniak L S, Zanni M V. Myocardial Steatosis Among Antiretroviral Therapy-Treated People With Human Immunodeficiency Virus Participating in the REPRIEVE Trial. J Infect Dis. 2020 Jul. 9; 222(Suppl 1):S63-S69. doi: 10.1093/infdis/jiaa245. PMID: 32645158; PMCID: PMC7347082, and Holloway C J, Ntusi N, Suttie J, Mahmod M, Wainwright E, Clutton G, Hancock G, Beak P, Tajar A, Piechnik S K, Schneider J E, Angus B, Clarke K, Dorrell L, Neubauer S. Comprehensive cardiac magnetic resonance imaging and spectroscopy reveal a high burden of myocardial disease in HIV patients. Circulation. 2013 Aug. 20; 128(8):814-22. doi: 10.1161/circulationaha.113.001719. Epub 2013 Jul. 1 PMID: 23817574 the entirety of the contents of which are incorporated by reference herein.

Myocardial steatosis in hypertensive heart disease is discussed in Sai E, Shimada K, Yokoyama T, Hiki M, Sato S, Hamasaki N, Maruyama M, Morimoto R, Miyazaki T, Fujimoto S, Tamura Y, Aoki S, Watada H, Kawamori R, Daida H. Myocardial triglyceride content in patients with left ventricular hypertrophy: comparison between hypertensive heart disease and hypertrophic cardiomyopathy. Heart Vessels. 2017 February; 32(2):166-174. doi: 10.1007/s00380-016-0844-8. Epub 2016 May 3. PMID: 27142065, the entirety of the contents of which are incorporated by reference herein.

Myocardial steatosis in pulmonary arterial hypertension is discussed in Brittain E L, Talati M, Fessel J P, Zhu H, Penner N, Calcutt M W, West J D, Funke M, Lewis G D, Gerszten R E, Hamid R, Pugh M E, Austin E D, Newman J H, Hemnes A R. Fatty Acid Metabolic Defects and Right Ventricular Lipotoxicity in Human Pulmonary Arterial Hypertension. Circulation. 2016 May 17; 133(20):1936-44. doi: 10.1161/circulationaha.115.019351. Epub 2016 Mar. 22. PMID: 27006481; PMCID: PMC4870107, the entirety of the contents of which are incorporated by reference herein.

Myocardial steatosis in coronary microvascular dysfunction is discussed in Wei J, Nelson M D, Szczepaniak E W, Smith L, Mehta P K, Thomson L E, Berman D S, Li D, Bairey Merz C N, Szczepaniak L S. Myocardial steatosis as a possible mechanistic link between diastolic dysfunction and coronary microvascular dysfunction in women. Am J Physiol Heart Circ Physiol. 2016 Jan. 1; 310(1):H14-9. doi: 10.1152/ajpheart.00612.2015. Epub 2015 Oct. 30. PMID: 26519031; PMCID: PMC4865076, the entirety of the contents of which are incorporated by reference herein.

Myocardial steatosis in generalized lipodystrophy is discussed in Nelson M D, Victor R G, Szczepaniak E W, Simha V, Garg A, Szczepaniak L S. Cardiac steatosis and left ventricular hypertrophy in patients with generalized lipodystrophy as determined by magnetic resonance spectroscopy and imaging. Am J Cardiol. 2013 Oct. 1; 112(7):1019-24. doi: 10.1016/j.amjcard.2013.05.036. Epub 2013 Jun. 22. PMID: 23800548; PMCID: PMC3779507, the entirety of the contents of which are incorporated by reference herein.

Myocardial steatosis and lipotoxicity in heart failure after cardiac transplantation in diabetics is discussed in Marfella R, Amarelli C, Cacciatore F, Balestrieri M L, Mansueto G, D'Onofrio N, Esposito S, Mattucci I, Salerno G, De Feo M, D'Amico M, Golino P, Maiello C, Paolisso G, Napoli C. Lipid Accumulation in Hearts Transplanted From Nondiabetic Donors to Diabetic Recipients. J Am Coll Cardiol. 2020 Mar. 24; 75(11):1249-1262. doi: 10.1016/j.jacc.2020.01.018. PMID: 32192650, the entirety of the contents of which are incorporated by reference herein.

### Administration of Compounds

The compounds and compositions may be provided in a dosage form and the dose may be provided by any suitable route or mode of administration. The dose may be provided orally, intravenously, enterally, parenterally, dermally, buccally, topically, transdermally, by injection, subcutaneously, nasally, pulmonarily, or with or on an implantable medical device (e.g., stent or drug-eluting stent or balloon equivalents).

The composition may be provided in one dose per day. The composition may be provided in multiple doses per day. The composition may be provided in two, three, four, five, six, eight, or more doses per day.

Relatedly, the compounds of the invention are useful for improving cardiac (mechanical) efficiency. A variety of definitions of cardiac efficiency exist in the medical literature. See, e.g. Schipke, J. D. Cardiac efficiency, Basic Res. Cardiol. 89:207-40 (1994); and Gibbs, C. L. and Barclay, C. J. Cardiac efficiency, Cardiovasc. Res. 30:627-634 (1995), incorporated herein by reference. One definition of cardiac mechanical efficiency is the ratio of external cardiac power to cardiac energy expenditure by the left ventricle. See Lopaschuk G. D., et al., Myocardial Fatty Acid Metabolism in Health and Disease, Phys. Rev. 90:207-258 (2010), incorporated herein by reference. Another definition is the ratio between stroke work (i.e. useful energy produced) and oxygen consumption, which ranges from 20-25% in the normal human heart. Visser, F., Measuring cardiac efficiency: is it useful? Hear Metab. 39:3-4 (2008), and also Knaapen P., et al., Myocardial energetics and efficiency: current status of the noninvasive approach, Circulation. 2007 Feb. 20; 115(7):918-27, both incorporated herein by reference. Another definition is the ratio of the stroke volume to mean arterial blood pressure. Any suitable definition of cardiac efficiency may be used to measure the effects of compounds of the invention.

Relatedly, the compounds of the invention are useful as mitotropic agents. Mitotropes are defined as pharmacological agents acting at the mitochondria which improve myocardial performance based on influencing energetics. See Psotka M. A., et al, Cardiac Calcitropes, Myotropes, and Mitotropes: JACC Review Topic of the Week, J Am Coll Cardiol. 2019 May 14; 73(18):2345-2353, doi: 10.1016/j.jacc.2019.02.051, PMID: 31072579, and DesJardin, J. T., Teerlink, J. R., Inotropic therapies in heart failure and cardiogenic shock: an educational review, Eur Heart J Acute Cardiovasc Care, 2021 Aug. 24; 10(6):676-686. doi: 10.1093/ehjacc/zuab047, PMID: 34219157, the entirety of the contents of both of which are incorporated by reference herein.

The compounds may include one or more atoms that are enriched for an isotope. For example, the compounds may have one or more hydrogen atoms replaced with deuterium or tritium. Isotopic substitution or enrichment may occur at carbon, sulfur, or phosphorus, or other atoms. The compounds may be isotopically substituted or enriched for a given atom at one or more positions within the compound, or the compounds may be isotopically substituted or enriched at all instances of a given atom within the compound.

In certain embodiments, methods of the invention include providing pharmaceutical compositions containing one or more of the compounds described above. A pharmaceutical composition containing a compound may be in a form suitable for oral use, for example, as tablets, troches, lozenges, fast-melts, aqueous or oily suspensions, dispersible powders or granules, emulsions, hard or soft capsules, syrups or elixirs. Compositions intended for oral use may be prepared according to any method known in the art for the manufacture of pharmaceutical compositions and such compositions may contain one or more agents selected from sweetening agents, flavoring agents, coloring agents and preserving agents, in order to provide pharmaceutically elegant and palatable preparations. Tablets contain the compounds in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. Preparation and administration of compounds is discussed in U.S. Pat. No. 6,214,841 and U.S. Patent Publication No. 2003/0232877, the contents of each of which are incorporated by reference herein.

Formulations for oral use may also be presented as hard gelatin capsules in which the compounds are mixed with an inert solid diluent, for example calcium carbonate, calcium phosphate or kaolin, or as soft gelatin capsules in which the compounds are mixed with water or an oil medium, for example peanut oil, liquid paraffin or olive oil.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions used in methods of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A compound represented by formula (X):

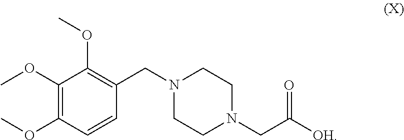 [SMILES: COc1ccc(CN2CCN(CC(=O)O)CC2)c(OC)c1OC]

2. A pharmaceutical composition comprising the compound of claim 1 and one or more pharmaceutically acceptable salts and/or excipients.

3. A method of treating cardiac dysfunction in a subject, the method comprising administering to the subject a composition comprising the compound of claim 1.

4. The method of claim 3, wherein the cardiovascular condition is selected from the group consisting of acute coronary syndrome, aneurysm, angina, anthracycline-induced cardiotoxicity, atherosclerosis, cardiac adiposity or steatosis, cardiac ischemia-reperfusion injury, cardiogenic shock, cardiomyopathy, cardiac lipotoxicity, cardioprotection, cardio-renal syndrome, cerebral vascular disease, chronic coronary syndromes, congenital heart disease, contrast nephropathy, coronary artery disease, coronary heart disease, coronary microvascular dysfunction, diabesity, diabetic cardiomyopathy, heart attack, heart disease, heart failure, cardiometabolic HFpEF, heart failure after cardiac transplantation including in diabetics, hibernating or stunned myocardium, hypertension, hypertensive heart disease, hypertrophic cardiomyopathy, ischemia with no obstructive coronary artery disease (INOCA), ischemia-reperfusion injury, ischemic heart disease, ischemic cardiomyopathy, lipotoxic cardiomyopathy, metabolic syndrome, microvascular angina, MINOCA, mitochondrial cardiomyopathies, myocardial dysfunction induced by anti-cancer drugs, myocardial infarction, non-ischemic cardiomyopathy, obesity cardiomyopathy, pericardial disease, pericardial (and/or epicardial) fat accumulation, peripheral arterial disease, pulmonary hypertension (PH)—including WHO group 1 (pulmonary arterial hypertension) group 2 (PH due to left heart disease) group 3 (PH due to lung disease) group 4 (chronic thromboembolic PH, CTEPH) and group 5 (PH due to unknown causes)—both primary and secondary, long COVID and post-acute COVID-19 cardiovascular sequelae, rheumatic heart disease, right heart failure, right ventricular failure, stroke, Takotsubo (stress) cardiomyopathy, transient ischemic attack(s), valvular heart disease, and vasospastic angina.
